(19) United States
(12) Patent Application Publication
(10) Pub. No.: US 2023/0093921 A1
(43) Pub. Date: Mar. 30, 2023

(54) ORIENTED AND COVALENT METHOD FOR IMMOBILIZING GLYCOPROTEIN AND ANTIBODY CHIP

(71) Applicant: National Tsing Hua University, Hsinchu City (TW)

(72) Inventors: Chun-Cheng Lin, Hsinchu City (TW); Avijit K. Adak, Hsinchu City (TW); Chen-Yu Fan, Hsinchu City (TW)

(73) Assignee: National Tsing Hua University, Hsinchu City (TW)

(21) Appl. No.: 17/570,334

(22) Filed: Jan. 6, 2022

(30) Foreign Application Priority Data
Aug. 31, 2021 (TW) 110132354

(51) Int. Cl. G01N 33/543; C07K 17/14

(52) U.S. Cl. CPC G01N 33/54306; C07K 17/14; G01N 33/54353; C07K 2317/41; C07K 2317/52

(57) ABSTRACT

The invention provides an oriented and covalent method for immobilizing a glycoprotein and an antibody on a chip. The method includes providing a silver-coated solid surface equipped with alkynes and cuprous oxide nanoparticles. The azido boronic acid tosyl probe is conjugated to the silver-coated solid surface by the cuprous oxide nanoparticles through the self-catalyzed azide-alkyne cycloaddition reaction. The glycan(s) of a glycoprotein or an antibody is provided to the boronic acid tosyl probe, and alcohol groups of the glycan(s) of the glycoprotein or the antibody and the boronic acid group of boronic acid tosyl probe form boronate ester. The nucleophilic residues on the glycoprotein or the antibody replace the tosyl group by SN2 reaction, so as to immobilize the glycoprotein or the antibody through the covalent bond formation.

### CROSS-REFERENCE TO RELATED APPLICATION

This application claims the priority benefit of Taiwan application serial no. 110132354, filed on Aug. 31, 2021. The entirety of the above-mentioned patent application is hereby incorporated by reference herein and made a part of this specification.

### BACKGROUND

### Technical Field

The disclosure relates to a method for immobilizing a glycoprotein and an antibody chip, and particularly relates to an oriented and covalent method for immobilizing a glycoprotein and an antibody chip.

### Description of Related Art

The antibody has tight and specific binding power with the epitope, so the antibody has been widely used in immunoaffinity separation, targeted therapy delivery, enzyme-linked immunosorbent assay (ELISA), inspection arrays, and other biomedical technology. The applications require the antibody to be bound onto a solid support as a carrier (for example, a microtiter plate, a nanoparticle, or a glass surface) while retaining the analyte binding activity.

Based on the chemical complexity of the antibody, the immobilization mechanism of the antibody is quite complex. The conventional antibody cross-linking method, such as the immobilized antibody prepared by physical adsorption, has relatively weak antibody binding ability. The substance contained in a complex specimen sample (for example, a blood sample) causes the antibody bound to boronic acid to dissociate, which affects the sensitivity of subsequent analysis of the antibody. If residues such as lysine, arginine, aspartate, and glutamate of the antibody are randomly selected for Schiff base or amide bond modification, there will be the disadvantage of random orientation, which can easily cause the antibody to lose the antigen binding activity.

Based on the above, developing a method for immobilizing a glycoprotein that can form a covalent bond with specific orientation, have high activity, have strong binding affinity, and increase the detection sensitivity is an important subject for current development of biomedical detection.

### SUMMARY

The disclosure provides an oriented and covalent method for immobilizing a glycoprotein and an antibody chip, which have high activity, have strong binding affinity, and increase the detection sensitivity.

The oriented and covalent method for immobilizing the glycoprotein of the disclosure includes the following steps. A silver-coated solid surface is provided. Multiple cuprous oxide nanoparticles are disposed on the silver-coated solid surface. Then, a solid surface (including the silver-coated solid surface and the cuprous oxide nanoparticles) is alkynylated. Next, an azido boronic acid tosyl probe is provided to the silver-coated solid surface. The boronic acid tosyl probe is bound to the solid surface by the cuprous oxide nanoparticles through self-catalyzed azide-alkyne cycloaddition (SAAC) reaction. Then, the glycoprotein is provided to the boronic acid tosyl probe. The glycoprotein may include an Fc fragment (crystallizable). An alcohol group of a glycan of the glycoprotein or a glycan chain of the Fc fragment and a boronic acid group of the boronic acid tosyl probe form organic boronate ester to immobilize the glycoprotein. Next, a nucleophilic residue on the glycoprotein is replaced with a tosyl group in the boronic acid tosyl probe by an S_{N}2 reaction, and the tosyl group is released from a terminal azide group to immobilize the glycoprotein through a covalent bond between the nucleophilic residue and the terminal azide group. Finally, the organic boronate ester is released.

In an embodiment of the disclosure, a structure of the azido boronic acid tosyl probe is represented by Formula (1) or Formula (1A):

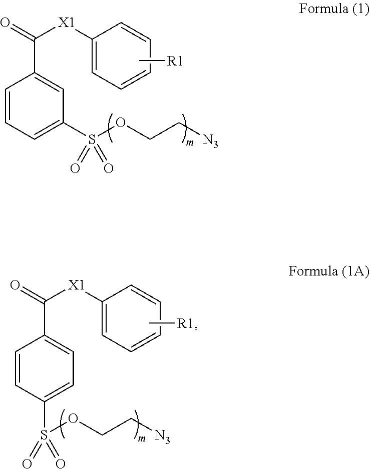

where in Formula (1) and Formula (1A), R1 is a boron-containing group, and a structure of an aromatic group with R1 group is represented by Formula (2), Formula (3), or Formula (4):

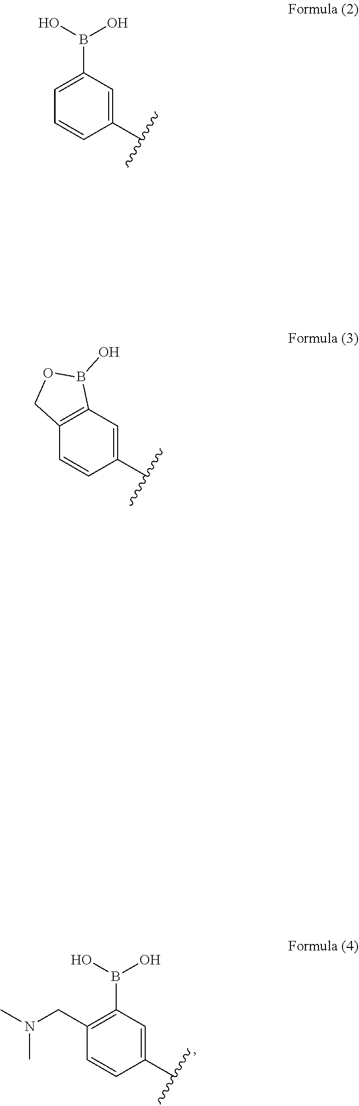

where X1 is NH,

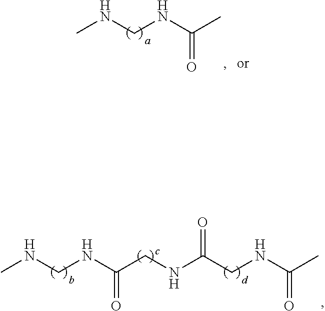

m is a positive integer from 1 to 8, a is a positive integer from 2 to 10, b is a positive integer from 2 to 10, c is a positive integer from 1 to 15, and d is a positive integer from 1 to 15.

In an embodiment of the disclosure, the structure of the boronic acid tosyl probe is represented by Formula (1-1), Formula (1-2), or Formula (1-3):

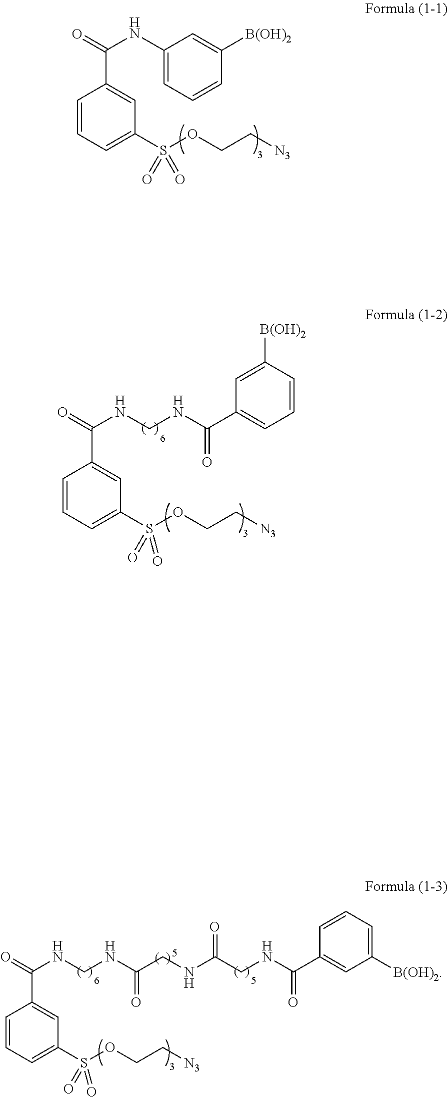

In an embodiment of the disclosure, a material of the silver-coated solid surface includes glass.

In an embodiment of the disclosure, a thickness of a silver coating layer of the silver-coated solid surface is 5 nm to 200 nm.

In an embodiment of the disclosure, alkynylation is to react the silver-coated solid surface disposed with the cuprous oxide nanoparticles with alkyne thiol whose structure is represented by Formula (A) or Formula (B) and thiol whose structure is represented by Formula

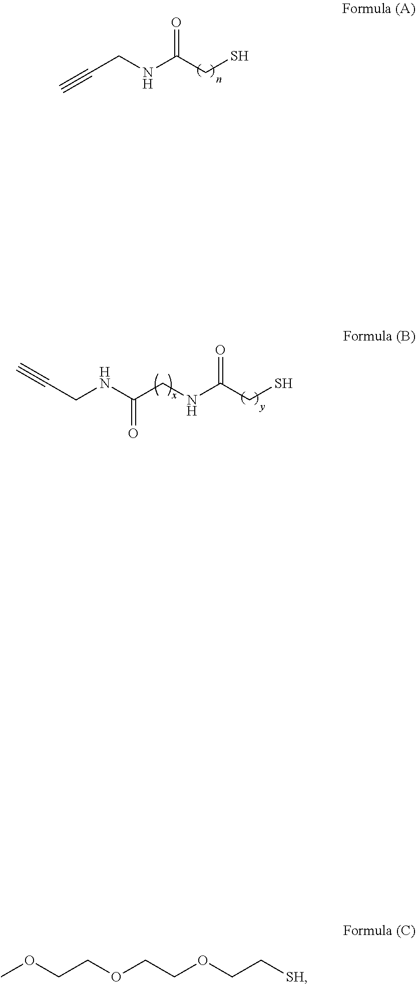

where in Formula (A), n is a positive integer from 5 to 15, and in Formula (B), x is a positive integer from 1 to 15, and y is a positive integer from 1 to 15.

In an embodiment of the disclosure, when providing the boronic acid tosyl probe to the silver-coated solid surface, azido-linked tri(ethylene glycol) is provided to the silver-coated solid surface at a same time, and azido-linked tri(ethylene glycol) is bonded to the cuprous oxide nanoparticles by self-catalyzed azide-alkyne cycloaddition reaction.

In an embodiment of the disclosure, the glycoprotein includes an antibody.

In an embodiment of the disclosure, releasing the organic boronate ester is performed through polyols.

The antibody chip of the disclosure uses the oriented and covalent method for immobilizing the glycoprotein.

Based on the above, the oriented and covalent method for immobilizing the glycoprotein of the disclosure uses the boronic acid tosyl probe, which not only enables the alcohol group of the glycan of the glycoprotein or the glycan chain of the Fc fragment and the boronic acid group of the boronic acid tosyl probe to form organic boronate ester, but also enables the nucleophilic residue on the glycoprotein to be replaced with the tosyl group in the boronic acid tosyl probe by the S_{N}2 reaction to form an oriented irreversible covalent bond. In addition, the oriented and covalent method for immobilizing the glycoprotein of the disclosure uses the silver-coated solid surface disposed with the cuprous oxide nanoparticles. The silver-coated solid surface has surface resonance to enhance a fluorescence signal (metal-enhanced fluorescence, MEF), and the cuprous oxide nanoparticles enable the boronic acid tosyl probe to be bound by self-catalyzed azide-alkyne cycloaddition (SAAC) reaction. Therefore, different from the conventional Cu(I)-catalyzed azide-alkyne cycloaddition (CuAAC) reaction, the use of additional copper ions is not required, and the issue of degraded boronate reactivity caused by Cu(I) can be solved. In this way, the activity can be effectively improved, the binding force can be strengthened, and the detection sensitivity can be increased.

### BRIEF DESCRIPTION OF THE DRAWINGS

FIG. 1A to FIG. 1G are schematic diagrams of an oriented and covalent method for immobilizing a glycoprotein according to an embodiment of the disclosure.

FIG. 2A and FIG. 2B are fluorescence detection diagrams of single-protein biomarkers for SAP and C-RP using a functional antibody microarray.

FIG. 3 is a fluorescence detection diagram for C-RP, SAP, and RCA₁₂₀ in blood serum using an oriented and covalent method for immobilizing a glycoprotein of the disclosure.

FIG. 4 is a fluorescence detection diagram of respectively using self-catalyzed azide-alkyne cycloaddition reaction and Cu(I)-catalyzed azide-alkyne cycloaddition.

### DETAILED DESCRIPTION OF DISCLOSED EMBODIMENTS

Hereinafter, embodiments of the disclosure will be described in detail. However, the embodiments are illustrative, and the disclosure is not limited thereto.

In the disclosure, the range represented by “a value to another value” is a general way to avoid listing all values in the range one by one in the specification. Therefore, the recitation of a specific value range covers any value in the value range and a smaller value range defined by any value in the value range, which is the same as writing the arbitrary value and the smaller value range in the specification.

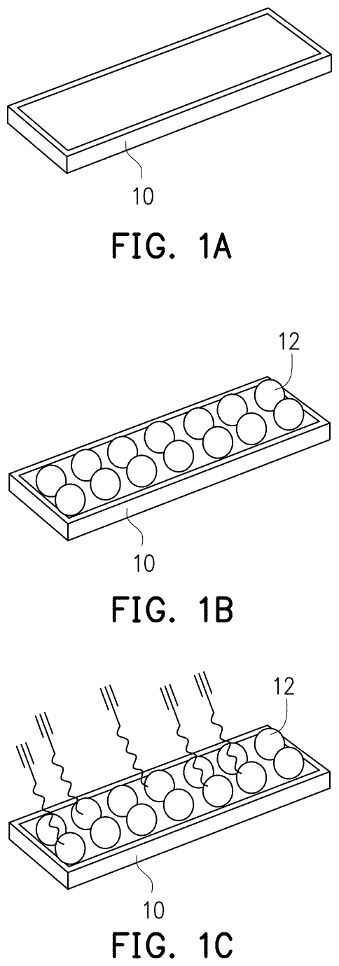

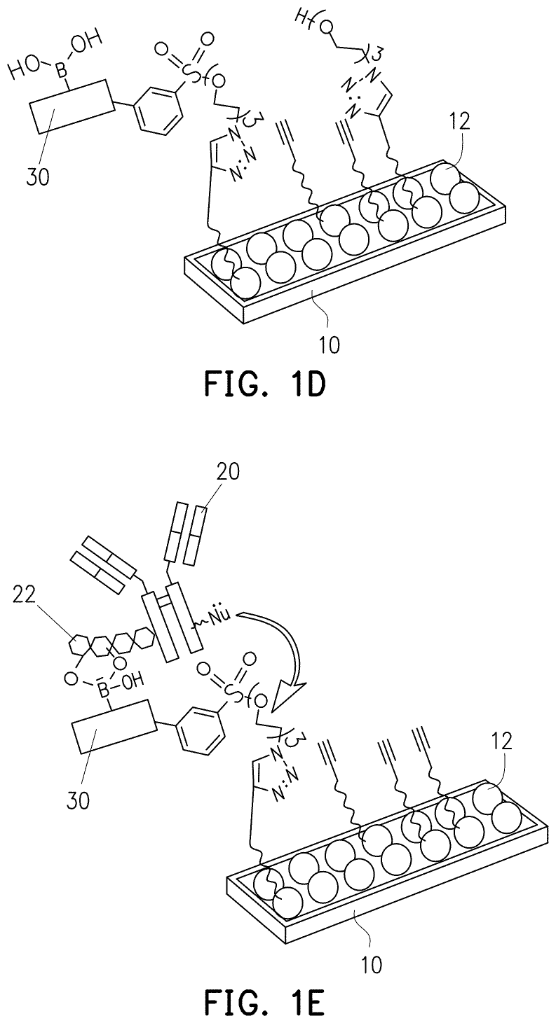

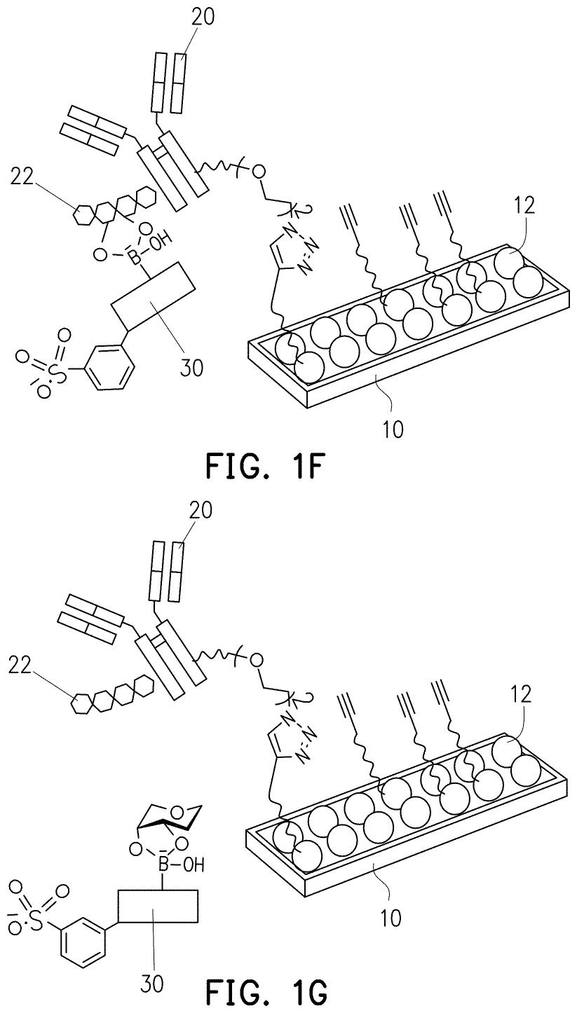

FIG. 1A to FIG. 1G are schematic diagrams of an oriented and covalent method for immobilizing a glycoprotein according to an embodiment of the disclosure.

Please refer to FIG. 1A. A silver-coated solid surface 10 is provided. In the embodiment, the thickness of a silver coating layer of the silver-coated solid surface 10 is 5 nm to 200 nm, and the material of the silver-coated solid surface 10 may include glass, but the disclosure is not limited thereto. In more detail, the manufacturing manner of the silver-coated solid surface 10 is, for example, using a silver nitrate reduction process to deposit silver on a solid surface. The concentration of silver nitrate is, for example, 0.01 M to 0.1 M, and the reaction temperature is, for example, 25° C. to 70° C.

Please refer to FIG. 1B. Multiple cuprous oxide nanoparticles 12 are disposed on the silver-coated solid surface 10. The size of the cuprous oxide nanoparticle 12 is, for example, between 10 nm and 100 nm. Afterwards, please refer to FIG. 1C. The silver-coated solid surface 10 and the cuprous oxide nanoparticles 12 are alkynylated for subsequent self-catalyzed azide-alkyne cycloaddition (SAAC) reaction. In more detail, alkynylation is, for example, reacting the silver-coated solid surface 10 disposed with the cuprous oxide nanoparticles 12 with alkyne thiol whose structure is represented by Formula (A) or Formula (B) and thiol whose structure is represented by Formula (C):

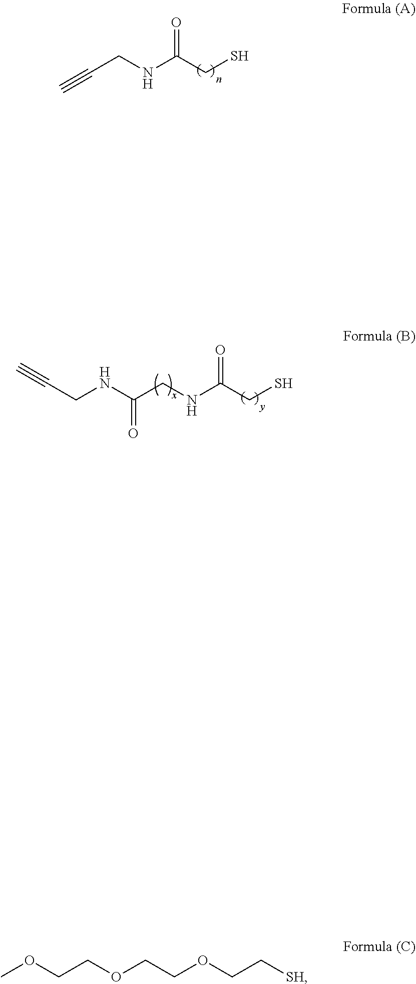

where in Formula (A), n is a positive integer from 5 to 15, and in Formula (B), x is a positive integer from 1 to 15, and y is a positive integer from 1 to 15.

In the embodiment, adding thiol represented by Formula (C) may adjust the degree of alkynylation of a surface. The molar ratio of the added amount of alkyne thiol represented by Formula (A) or Formula (B) to thiol represented by Formula (C) is, for example, 0.01 to 100. In FIG. 1C, for concise description, some chemical structural formulas are omitted, and only alkynylation is shown as a schematic illustration.

Please refer to FIG. 1D. A boronic acid tosyl probe is provided to the silver-coated solid surface 10. The boronic acid tosyl probe is bound to the cuprous oxide nanoparticles 12 by self-catalyzed azide-alkyne cycloaddition (SAAC) reaction. In the embodiment, when providing the boronic acid tosyl probe to the silver-coated solid surface 10, azido-linked tri(ethylene glycol) may also be provided to the silver-coated solid surface at the same time. Azido-linked tri(ethylene glycol) may also be bound to the cuprous oxide nanoparticles 12 by self-catalyzed azide-alkyne cycloaddition reaction. In addition to consuming remaining alkynyl groups, the hydrophilicity of a surface may be further improved through adding azido-linked tri(ethylene glycol). The structure of azido-linked tri(ethylene glycol) is shown below:

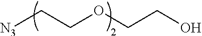

In the embodiment, the structure of the boronic acid tosyl probe may be represented by Formula (1) or Formula (1A):

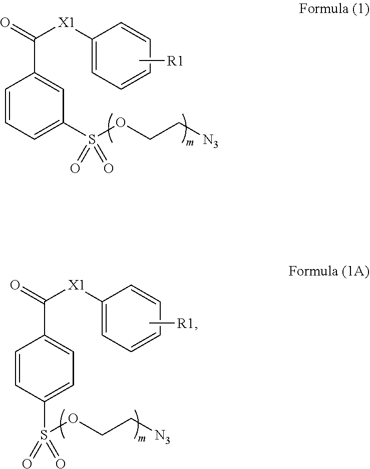

where in Formula (1) and Formula (1A), R1 is a boron-containing group, and the structure of an aromatic group with R1 group may be represented by Formula (2), Formula (3), or Formula (4):

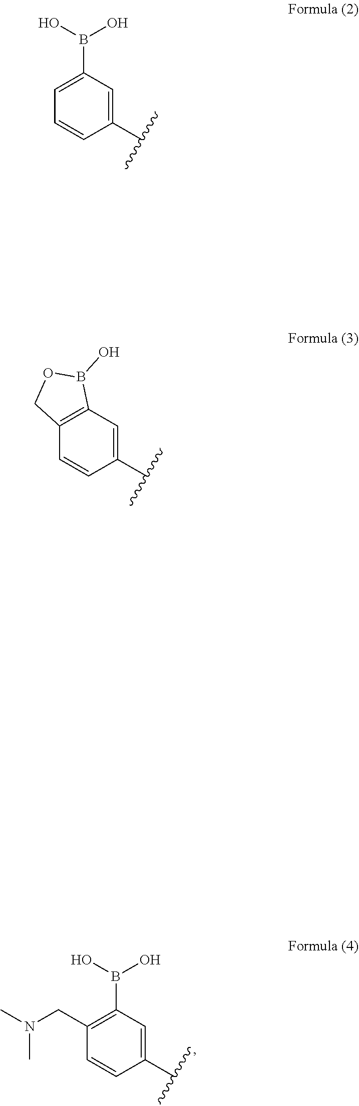

where X1 is NH,

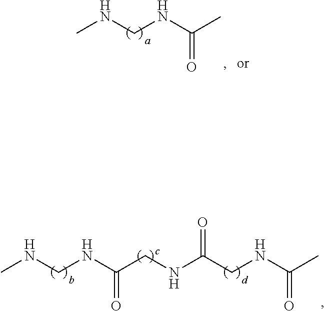

m is, for example, a positive integer from 1 to 8, a is, for example, a positive integer from 2 to 10, b is, for example, a positive integer from 2 to 10, c is, for example, a positive integer from 1 to 15, and d is, for example, a positive integer from 1 to 15.

In FIG. 1D and the following drawings, for concise description, some chemical structural formulas are omitted, and only a linker 30 is shown as a schematic illustration.

The specific structure of the boronic acid tosyl probe may be represented by Formula (1-1), Formula (1-2), or Formula (1-3):

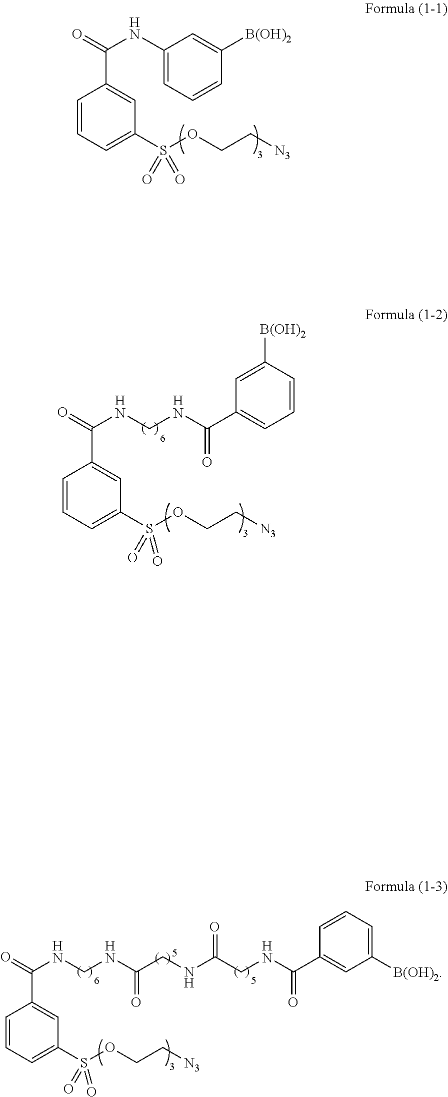

Please refer to FIG. 1E and FIG. 1F. A glycoprotein is provided to the boronic acid tosyl probe. The glycoprotein may include an Fc fragment (crystallizable). In the embodiment, the glycoprotein is, for example, an antibody 20, but the disclosure is not limited thereto. An alcohol group of a glycan chain 22 of the Fc fragment of the antibody 20 and a boronic acid group of the boronic acid tosyl probe form organic boronate ester to immobilize the glycoprotein (for example, the antibody 20 in the embodiment) onto the solid surface (the silver-coated solid surface 10 and the cuprous oxide nanoparticles 12). In more details, the glycoprotein may be obtained by genetic engineering and glycoprotein engineering. Therefore, the glycoprotein may be an Fc-fusion glycoprotein, which includes the Fc fragment with the glycan chain to implement the oriented and covalent method for immobilizing the glycoprotein of the disclosure. However, the disclosure is not limited thereto. The glycoprotein may not include the Fc fragment, that is, the alcohol group of a glycan of the glycoprotein or the glycan chain of the Fc fragment may both form organic boronate ester with the boronic acid group of the boronic acid tosyl probe to immobilize the glycoprotein. A nucleophilic residue (Nu:) on the glycoprotein (for example, the antibody 20 in the embodiment) is replaced with a tosyl group in the boronic acid tosyl probe by an S_{N}2 reaction, and the tosyl group is released to immobilize the glycoprotein (for example, the antibody 20 in the embodiment) through a covalent bond between the nucleophilic residue and a terminal azide group. The site where the covalent bond is formed is close to a region where boronate ester is formed, so the method has orientation selectivity.

Next, please refer to FIG. 1G. Organic boronate ester is released. In the embodiment, the release of the organic boronate ester is, for example, performed through polyols.

Polyols may include glycerol, sorbitol, mannitol, or polyethylene glycol, but the disclosure is not limited thereto. In this way, by the oriented and covalent method for immobilizing the glycoprotein of the disclosure, when the glycoprotein is, for example, the antibody, the binding force of the antibody can be improved, and the dissociation of the antibody caused by the substance in a complex specimen sample can be avoided. Therefore, the oriented and covalent method for immobilizing the glycoprotein of the disclosure is suitable for further detection of an antigen in a blood sample, which can effectively improve the sensitivity of subsequent analysis of the antibody.

The disclosure also provides an antibody chip using the oriented and covalent method for immobilizing the glycoprotein.

Hereinafter, the oriented and covalent method for immobilizing the glycoprotein of the above embodiment will be explained in detail through an experimental example. However, the following experimental example is not intended to limit the disclosure.

### Experimental Example

In order to prove that the oriented and covalent method for immobilizing the glycoprotein proposed by the disclosure can effectively improve the activity, strengthen the binding force, and increase the detection sensitivity, the following experimental example is provided.

It should be noted that since the oriented and covalent method for immobilizing the glycoprotein has been described in detail above, the following description of the oriented and covalent method for immobilizing the glycoprotein is omitted for convenience of explanation.

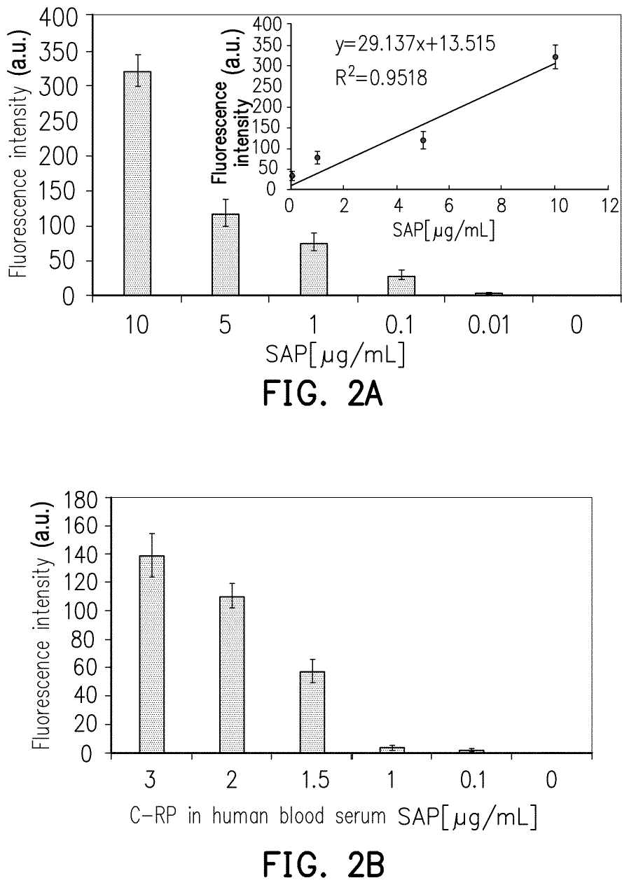

FIG. 2A and FIG. 2B are fluorescence detection diagrams of single-protein biomarkers for SAP and C-RP using a functional antibody microarray.

The oriented and covalent method for immobilizing the glycoprotein of the disclosure was used to measure two single biomarkers SAP and C-RP. Different concentrations of SAP (0.01 μ/mL, 0.1 μg/mL, 1 μg/mL, 5 μg/mL, and 10 μg/mL) were reacted together with an anti-SAP antibody produced using the oriented and covalent method for immobilizing the glycoprotein of the disclosure. Then, biotinylated anti-SAP pAb (1 μg/mL) was used to assess the presence of a binding protein, and fluorescent Cy3-labeled streptavidin (10 μg/mL) was then used. FIG. 2A shows the corresponding fluorescence intensity detection diagram, and the lowest analyte concentration that could generate a detection signal was 1 μg/mL. C-RP with concentrations of 0.1 μg/mL, 1.0 μg/mL, 1.5 μg/mL, 2.0 μg/mL, and 3.0 μg/mL were analyzed, and were reacted together with an anti-hC-RP antibody chip produced using the oriented and covalent method for immobilizing the glycoprotein of the disclosure. Then, a biotinylated C-RP detection antibody (1/100 dilution) was used, and fluorescent Cy3-labeled streptavidin was then used. FIG. 2B shows the corresponding fluorescence intensity detection diagram, and the sensitivity was about 1.4 μg/mL (˜57 nM), which is within the critical concentration range for cardiovascular risk assessment. Therefore, the achievable sensitivity of the oriented and covalent method for immobilizing the glycoprotein of the disclosure is suitable for direct detection of the biomarkers SAP and C-RP.

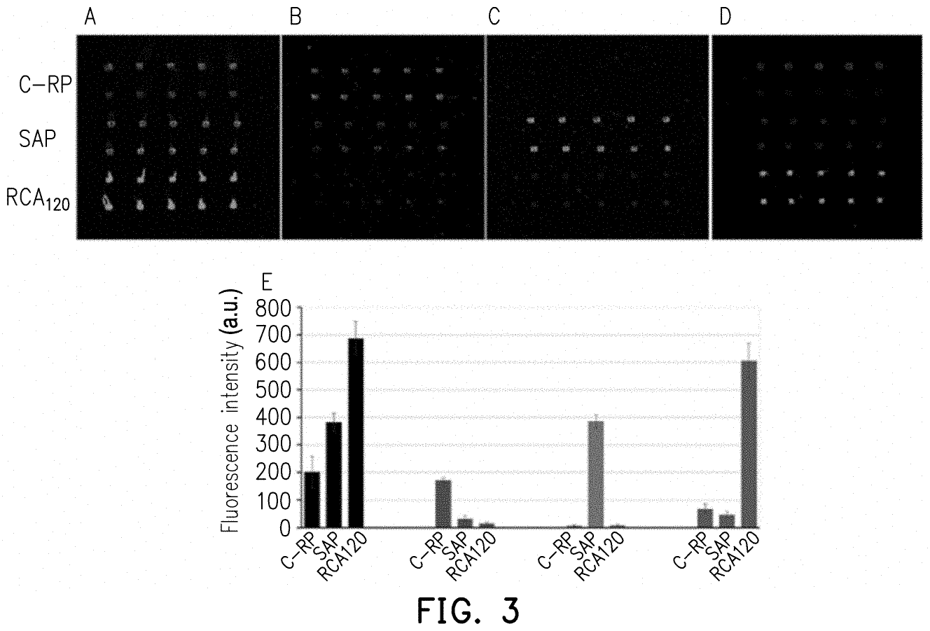

FIG. 3 is a fluorescence detection diagram for C-RP, SAP, and RCA₁₂₀ in blood serum using an oriented and covalent method for immobilizing a glycoprotein of the disclosure.

B, C, and D of FIG. 3 represent fluorescence images, which respectively depict three sets of captured antibody array points, and indicate that a specific protein is successfully identified. In addition, A of FIG. 3A illustrates the simultaneous capture and detection of three targets. As shown in E of FIG. 3, as the concentration increases, a fluorescent signal is generated due to the capture of more analytes, resulting in a higher fluorescence intensity on the spots of the array. Therefore, it can be known that the oriented and covalent method for immobilizing the glycoprotein of the disclosure can analyze multiple targets in a complex biological sample at the same time.

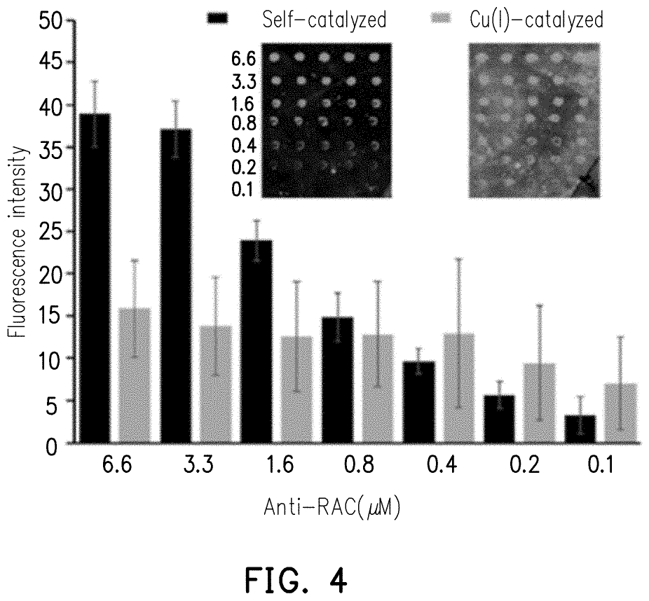

FIG. 4 is a fluorescence detection diagram of respectively using self-catalyzed azide-alkyne cycloaddition reaction and Cu(I)-catalyzed azide-alkyne cycloaddition.

As shown in FIG. 4, the use of Cu(I)-catalyzed azide-alkyne cycloaddition shows a strong background signal, which indicates that phenylboronate may have undergone Cu(I)-mediated decomposition into phenols or benzenes, thereby causing strong non-specific protein adsorption. In contrast, the use of the self-catalyzed azide-alkyne cycloaddition reaction as in the oriented and covalent method for immobilizing the glycoprotein of the disclosure can reduce the background signal and avoid non-specific protein adsorption.

In summary, the oriented and covalent method for immobilizing the glycoprotein of the disclosure uses the boronic acid tosyl probe, which not only enables the alcohol group of the glycan of the glycoprotein or the glycan chain of the Fc fragment and the boronic acid group of the boronic acid tosyl probe to form organic boronate ester, but also enables the nucleophilic residue on the glycoprotein to be replaced with the tosyl group in the boronic acid tosyl probe by the S_{N}2 reaction to form an oriented irreversible covalent bond and have higher binding specificity. In addition, the oriented and covalent method for immobilizing the glycoprotein of the disclosure uses the silver-coated solid surface disposed with the cuprous oxide nanoparticles. The silver-coated solid surface has surface resonance to enhance a fluorescence signal (metal-enhanced fluorescence, MEF), and the cuprous oxide nanoparticles enable the boronic acid tosyl probe to be bound by self-catalyzed azide-alkyne cycloaddition (SAAC) reaction. Therefore, different from the conventional Cu(I)-catalyzed azide-alkyne cycloaddition (CuAAC) reaction, the use of additional copper ions is not required, and the issue of degraded boronate reactivity caused by Cu(I) can be solved. In this way, the glycoprotein can resist dissociation in a complex sample (for example, a blood sample) while effectively improving the activity, strengthening the binding force, and increasing the detection sensitivity, and the orientation can also be taken into account.

What is claimed is:

1. An oriented and covalent method for immobilizing a glycoprotein, comprising:
providing a silver-coated solid surface, wherein a plurality of cuprous oxide nanoparticles are disposed on the silver-coated solid surface;
alkynylating the silver-coated solid surface and the cuprous oxide nanoparticles;
providing a boronic acid tosyl probe to the silver-coated solid surface, wherein the boronic acid tosyl probe is bound to the silver-coated solid surface by self-catalyzed azide-alkyne cycloaddition (SAAC) reaction;
providing the glycoprotein to the boronic acid tosyl probe, wherein an alcohol group of a glycan of the glycoprotein or a glycan chain of an Fc fragment and a boronic acid group of the boronic acid tosyl probe form organic boronate ester to immobilize the glycoprotein;
replacing a nucleophilic residue on the glycoprotein with a tosyl group in the boronic acid tosyl probe by an S_{N}2 reaction, and releasing the tosyl group to immobilize the glycoprotein through a covalent bond between the nucleophilic residue and a terminal azide group; and
releasing the organic boronate ester.

2. The oriented and covalent method for immobilizing the glycoprotein according to claim 1, wherein a structure of the boronic acid tosyl probe is represented by Formula (1) or Formula (1A): 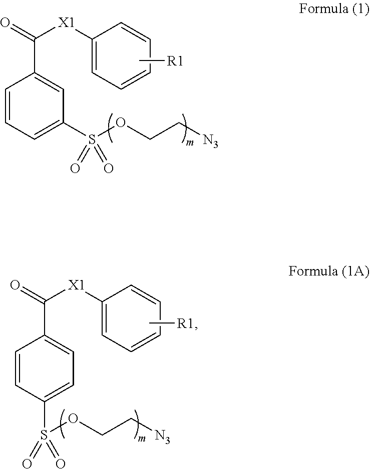
where in Formula (1) and Formula (1A), R1 is a boron-containing group, and a structure of an aromatic group with R1 group is represented by Formula (2), Formula (3), or Formula (4): 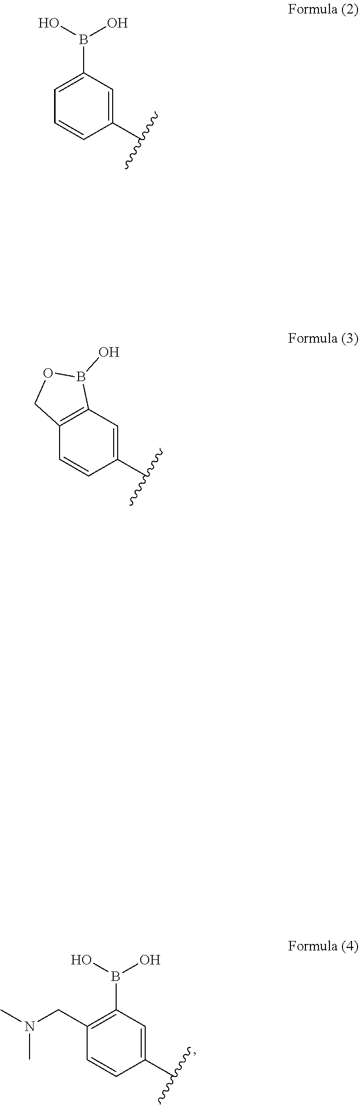
where X1 is NH, 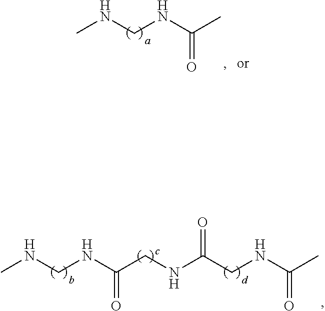
m is a positive integer from 1 to 8, a is a positive integer from 2 to 10, b is a positive integer from 2 to 10, c is a positive integer from 1 to 15, and d is a positive integer from 1 to 15.

3. The oriented and covalent method for immobilizing the glycoprotein according to claim 2, wherein the structure of the boronic acid tosyl probe is represented by Formula (1-1), Formula (1-2), or Formula (1-3):

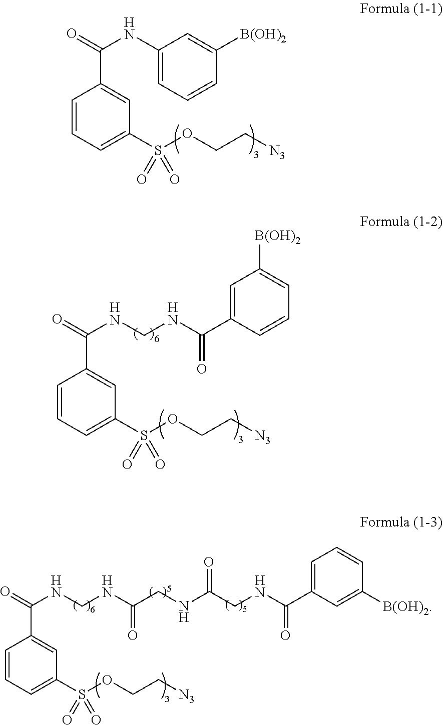

4. The oriented and covalent method for immobilizing the glycoprotein according to claim 1, wherein a material of the silver-coated solid surface comprises glass.

5. The oriented and covalent method for immobilizing the glycoprotein according to claim 1, wherein a thickness of a silver coating layer of the silver-coated solid surface is 5 nm to 200 nm.

6. The oriented and covalent method for immobilizing the glycoprotein according to claim 1, wherein alkynylation is to react the silver-coated solid surface disposed with the cuprous oxide nanoparticles with alkyne thiol whose structure is represented by Formula (A) or Formula (B) and thiol whose structure is represented by Formula (C): 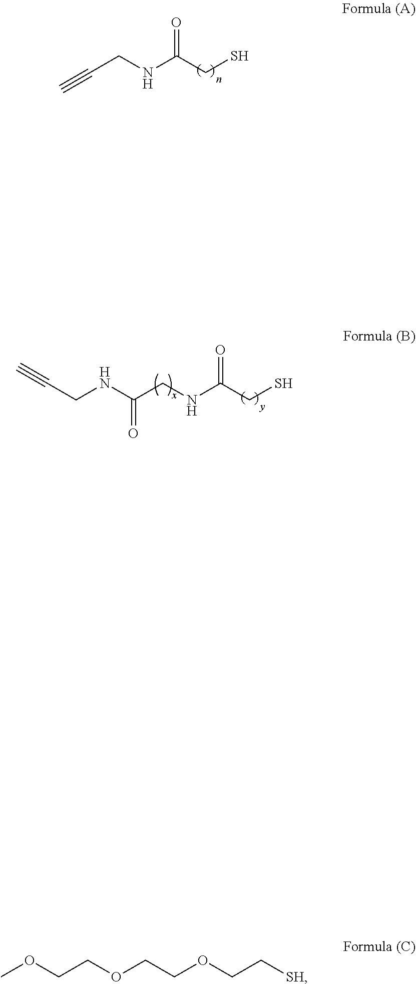
where in Formula (A), n is a positive integer from 5 to 15, and in Formula (B), x is a positive integer from 1 to 15, and y is a positive integer from 1 to 15.

7. The oriented and covalent method for immobilizing the glycoprotein according to claim 1, wherein when providing the boronic acid tosyl probe to the silver-coated solid surface, azido-linked tri(ethylene glycol) is provided to the silver-coated solid surface at a same time, and azido-linked tri(ethylene glycol) is bonded to the cuprous oxide nanoparticles by self-catalyzed azide-alkyne cycloaddition reaction.

8. The oriented and covalent method for immobilizing the glycoprotein according to claim 1, wherein the glycoprotein comprises an antibody.

9. The oriented and covalent method for immobilizing the glycoprotein according to claim 1, wherein releasing the organic boronate ester is performed through polyols.

10. An antibody chip, using the oriented and covalent method for immobilizing the glycoprotein according to claim 1.
